(19) United States
(12) Patent Application Publication
(10) Pub. No.: US 2019/0101551 A1
(43) Pub. Date: Apr. 4, 2019

(54) Real-time and Continuous Measurement in Vivo Using Aptamer-Based Biosensors

(71) Applicant: The Regents of the University of California, Oakland, CA (US)

(72) Inventors: Kevin Plaxco, Santa Barbara, CA (US); Netzahualcoyotl Arroyo Curras, Santa Barbaa, CA (US); Jacob Somerson, Santa Barbara, CA (US)

(73) Assignee: The Regents of the University of California, Oakland, CA (US)

(21) Appl. No.: 16/086,590

(22) PCT Filed: Jan. 27, 2017
(86) PCT No.: PCT/US17/15225

§ 371 Date: Sep. 19, 2018

Related U.S. Application Data
(60) Provisional application No. 62/311,190, filed on Mar. 21, 2016.

(51) Int. Cl. G01N 33/94; G01N 33/543; A61B 5/1473; A61B 5/00; G01N 33/50

(52) U.S. Cl. CPC G01N 33/94; G01N 33/54373; G01N 33/5088; A61B 5/4839; A61B 5/6847; A61B 5/14735

(57) ABSTRACT

The invention encompasses novel sensor designs that can operate in complex samples like whole blood. The use of protective filtering membranes prevents fouling and erroneous signal drift in sensors such as aptamer based electrochemical sensors. In one aspect, the invention encompasses implantable sensors that can be deployed to the circulatory system of an animal where they can accurately and continuously measure the concentration of a target species, such as a drug, with very short resolution times, for extended periods without signal drift. These sensor designs and associated methods provide a means of accurately dosing animals based on real-time monitoring of drugs and other chemical markers and biomarkers.

### CROSS-RELATED APPLICATIONS

This application claims the benefit of priority to U.S. Provisional Application Ser. No. 62/311,190, entitled “Aptamer Based Biosensor for Effective In Vivo Measurement of Analytes,” filed Mar. 21, 2016, the contents of which are hereby incorporated by reference.

### STATEMENT REGARDING FEDERALLY SPONSORED RESEARCH OR DEVELOPMENT

This invention was made with government support under grant number F31 CA183385-03 awarded by the National Institutes of Health and W911NF-09-D-0001 awarded by the Army Research Laboratory. The government has certain rights in the invention.

### BACKGROUND OF THE INVENTION

The availability of versatile and convenient sensors supporting the continuous, real-time measurement of specific molecules directly in the body could prove transformative in research and in medicine. As a research tool, such an advance would allow the in vivo concentrations of drugs, metabolites, hormones, and other biomarkers to be measured with high precision. Additionally, such an advance would facilitate “therapeutic drug monitoring,” in which dosing is personalized using a patient's directly-measured (rather than crudely and indirectly estimated) metabolism. By permitting the continuous monitoring of biomarkers such a technology could likewise provide a new and highly-detailed window into health status. Finally, the real-time measurement of specific molecules in the body would advance drug delivery by enabling feedback-controlled dosing, in which the delivery of drugs is adjusted in real time based on their concentration in the body or on the body's molecular-level response to treatment. This real-time, feedback-controlled drug delivery would provide new routes by which drugs with dangerously narrow therapeutic windows or complex optimal dosing regimens can be administered safely and efficiently.

Although technologies already exist for the continuous or near-continuous measurement of a small number of metabolites [e.g., glucose, lactate] and neurotransmitters [e.g., dopamine, serotonin, glutamate, and acetylcholine] in vivo, these approaches all rely on the specific chemical reactivities of their targets (e.g., the redox chemistry of the analyte or its ability to be oxidized by a specific enzyme). Because of their dependence on reactivity, these technologies are not generalizable to the detection of many other physiologically or clinically important molecules, and there remains an ongoing need in the art for technologies that support the continuous detection of specific molecules irrespective of their reactivity.

Serious technical hurdles stand in the way of realizing this goal of continuous real-time detection of specific molecules in the body. First, to support continuous measurements, a sensor cannot rely on batch processing, such as wash or separation steps. Second, to support in vivo measurements, a sensor cannot use exogenously-added reagents and must remain stable against prolonged exposure to blood or interstitial fluids in vivo. To date, the vast majority of molecular detection strategies have failed to meet one or both of these critical challenges. Chromatography, mass spectrometry, and immunochemistry, for example, are complex, multistep batch processes requiring wash steps, separation steps, and/or sequential reagent additions, hindering their ability to perform continuous measurements. Conversely, whereas biosensors based on surface plasmon resonance, quartz crystal micro-balances, field-effect transistors, and microcantilevers all support continuous, real-time operation, each fails when challenged in blood (much less in vivo) due to their inability to discriminate between the specific binding of their target and the nonspecific adsorption of proteins and cells.

Electrochemical aptamer-based (EAB) sensors provide a sensing platform adaptable to the detection of a wide range of molecular targets. These sensors comprise a conformation-changing aptamer probe that is covalently attached via one terminus to an integrated electrode and modified at the other terminus with a redox reporter. Upon binding to its target molecule, the probe undergoes a conformational rearrangement that modulates the redox current and generates an electrochemical signal. Since the conformational change is reversible, the probe enables continuous, sensitive, label-free detection with rapid kinetics and highly-specific binding of target species. However, as with other types of sensors, EAB sensors are subject to fouling after prolonged exposure to whole blood and other complex samples, precluding their use directly in vivo.

Previously, use of a continuous diffusion filter was provided as a solution for the problem EAB sensor fouling, as described in Ferguson et al., Real-time, aptamer-based tracking of circulating therapeutic agents in living animals, Sci Transl Med. 2013 Nov. 27; 5(213). That device employed a microfluidic filter using two stacked laminar flows: a bottom flow of blood continuously drawn via a jugular catheter from the animal and draining into a waste chamber, and a flow of buffer stacked on top of this first layer and in permanent contact with the relevant EAB sensor. The buffer sheath acted as a continuous-flow diffusion filter, allowing for rapid diffusion of small-molecule targets to the sensor while preventing the approach of (much more slowly diffusing) blood cells. While successful in avoiding fouling, the continuous diffusion filter suffers from substantial limitations. The continuous diffusion filter is only usable ex vivo, suffers from a time lag, requires continuous blood draw, and can only be used to measure molecules in blood because other bodily fluids cannot easily and continuously be withdrawn. The continuous diffusion filter device is also complex, requiring a pump and buffer and waste reserves. Additionally, the device is sensitive to mechanical shock disrupting the laminar flow and thus cannot be deployed in awake, freely moving animals.

In sum, to date there is no platform that satisfactorily addresses the various obstacles which prevent the continuous in vivo detection of clinically relevant target molecules. Accordingly, there remains an ongoing need in the art for technologies that support the continuous real-time detection of specific molecules in vivo.

### SUMMARY OF THE INVENTION

The inventors of the present disclosure have advantageously developed novel sensor designs that can function in living animals for long time periods with limited fouling or degradation of sensor sensitivity. The invention encompasses the use of porous materials to encase sensors, such as EAB sensors, to prevent their fouling by non-target species present in complex samples such as blood. The porous filters may comprise various materials, for example polysulfone. The use of such filters is demonstrated herein to enable the continuous and accurate measurement of analytes in vivo for extended periods of time.

In one aspect, the invention provides a method of preventing the fouling of sensors exposed to complex samples such as blood. In another aspect, the invention provides novel filters that may be applied to sensors to prevent their fouling. In another aspect, the invention provides an improvement to EAB sensors that enables their deployment in vivo. In another aspect, the invention provides a novel sensor design suitable for continuous in vivo use. In another aspect, the invention provides novel drug delivery methods and associated devices.

### BRIEF DESCRIPTION OF THE DRAWINGS

FIGS. 1A and 1B. FIGS. 1A and 1B are diagrams, which depict the configuration and operating principal of a standard EAB sensor. FIG. 1A depicts a sensor comprising an aptamer wherein the target species is unbound. FIG. 1B depicts the EAB sensor when the target is bound to the aptamer.

FIG. 2. FIG. 2 is a diagram depicting an exemplary sensor of the invention.

FIG. 3. FIG. 3 depicts the signal response of EAB sensors directed to the detection of aminoglycoside. The plot depicts the response of conventional EAB aminoglycoside sensors (labeled “No membrane”) and that of modified EAB aminoglycoside sensors having filtering encasements (labeled “Membrane”) in flowing, undiluted whole blood in vitro over many hours. Error bars are standard deviation of the mean of results collected from multiple independently fabricated devices.

FIG. 4. FIG. 4 depicts the continuous measurement of the antibiotic tobramycin by a sensor of the invention in the bloodstream of an anesthetized rat. Shown are data collected on a living rat given two sequential 20 mg/kg intravenous injections of the drug (at time denoted by vertical dotted lines).

### DETAILED DESCRIPTION OF THE INVENTION

The inventions disclosed herein encompass various novel devices and methods of use. The inventions disclosed herein include the use of novel protective membranes that surround and protect sensor surfaces from non-specific binding and degradation of sensor function.

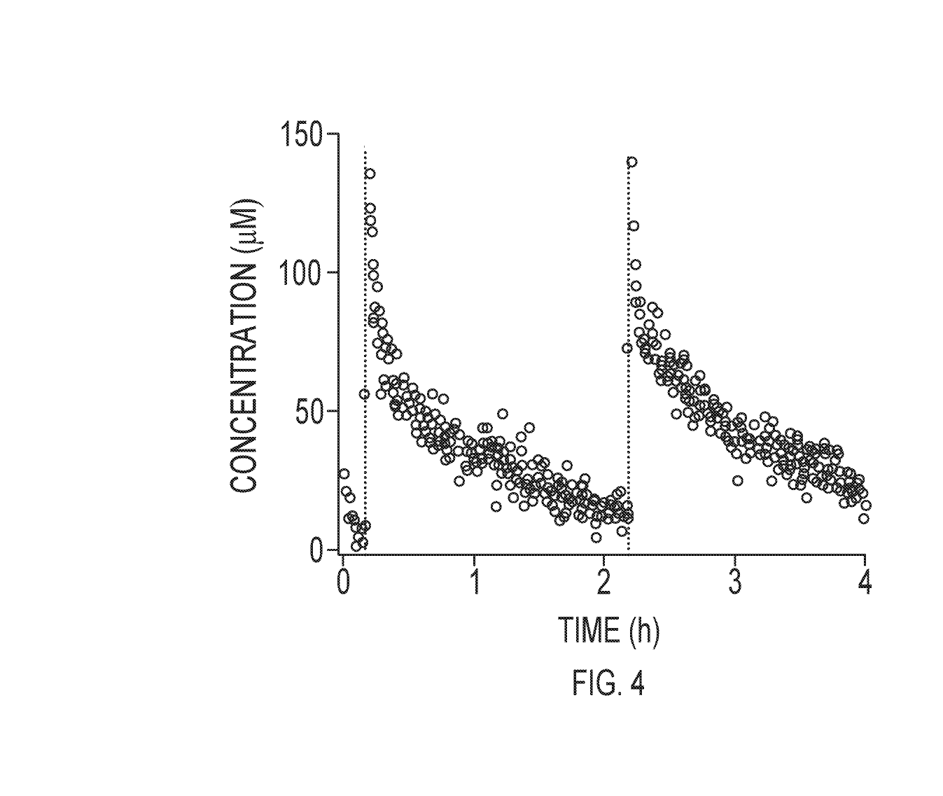

The novel sensors of the invention have been successfully tested for extended time periods in live animals, where they remained highly sensitive and reliable despite prolonged exposure to whole blood in vivo. FIG. 4 depicts the continuous measurement of the antibiotic tobramycin by a sensor of the invention in the bloodstream of an anesthetized rat. Shown are data collected on a living rat given two sequential 20 mg/kg intravenous injections of the drug (at times denoted by vertical dotted lines), demonstrating the sensor's ability to accurately track target species concentration at short time scales, for extended periods of time

The ability to perform continuous measurement of specific molecules in the body provides the art with new tools for the study of physiology and pharmacokinetics and with improved methods of drug delivery. Having a resolution time of a few seconds, the sensors of the invention have vastly improved time resolution compared to that of traditional pharmaco-kinetic methods, sufficient to measure the kinetics with which drugs distribute following intravenous injection, a pharmacokinetic phase that has rarely if ever been previously measured. Indeed, the precision of measurements achieved by the systems of the invention is sufficient not only to robustly identify animal-to-animal pharmacokinetic variability, but even variability within a single animal over the course of a few hours.

The description provided herein will make reference to certain measurements and samples in or from a “patient” or “animal.” It will be understood that such terms are non-limiting and may refer to any living organism. The living organism may be of any species, including bacterial and yeast organisms, plants, animals and humans. In one aspect, the methods of the invention are directed to humans, including human patients and users. In one aspect, the methods of the invention are applied to animal species, including veterinary patients or test animals.

The various elements of the invention are described next.

Sensors. The devices and methods described herein encompass the use of sensors. A sensor, as used herein, is a device that is capable of measuring the concentration of a target species in solution. The target species may be any inorganic or organic molecule, for example: a small molecule drug, a metabolite, a hormone, a peptide, a protein, a carbohydrate, a nucleic acid, or any other composition of matter. The target species may comprise a drug. The drug may be of any type, for example, including drugs for the treatment of cardiac system, the treatment of the central nervous system, that modulate the immune system, that modulate the endocrine system, an antibiotic agent, a chemotherapeutic drug, or an illicit drug. The target species may comprise a naturally-occurring factor, for example a hormone, metabolite, growth factor, neurotransmitter, etc. The target species may comprise any other species of interest, for example, species such as pathogens (including pathogen induced or derived factors), nutrients, and pollutants.

The sensors of the invention comprise various components. A first component is the sensing assembly. The sensing assembly comprises a sensing element and a filtering encasement. The sensing element is that portion of the sensor wherein binding of the target species occurs and wherein such binding generates a measurable signal. For example, in the case of an EAB sensing element, the portion of the electrode functionalized with aptamers is the sensing element. The sensing assembly further comprises a filtering encasement, as described below. For most electrochemical sensors, the sensing assembly will further comprise one or more reference electrodes.

The sensing assembly will further comprise wires or other electrically conductive elements which connect the sensing electrode and any reference electrodes to power supplies, voltage regulators, and other control elements which operate the sensing element. The sensing assembly may further comprise structures that house or support the various elements of the sensing assembly, holding them in place to ensure proper operation.

In addition to the sensing assembly, sensors of the invention will further comprise ancillary components which aid in the operation of the sensor. Sensor ancillary components may include power supplies (e.g., batteries) or connectors for power outlets. Sensor ancillary components further include controllers which generate currents and or voltages in the working and reference electrodes within the proper operating parameters. Sensor ancillary components further include readout circuitry, data collection, and storage components, e.g. processors and data storage drives that enable collection of signals from the sensing element, processing of such signals, recording and storage of such signals, or export of such signals to other data processing or data storage devices.

Sensors may be used in combination with housing elements that neatly contain and protect the sensor. Sensors may be used in combination with elements that hold the sensor in place on the body of the patient, for example collars, bracelets, straps, adhesives, dressings, etc.

The sensor may be a sensor of any type. In one embodiment, the sensor is an EAB sensor. Other exemplary sensor designs include surface plasmon resonance sensors, quartz crystal microbalance sensors, field-effect transistors, and microcantilever-based sensors.

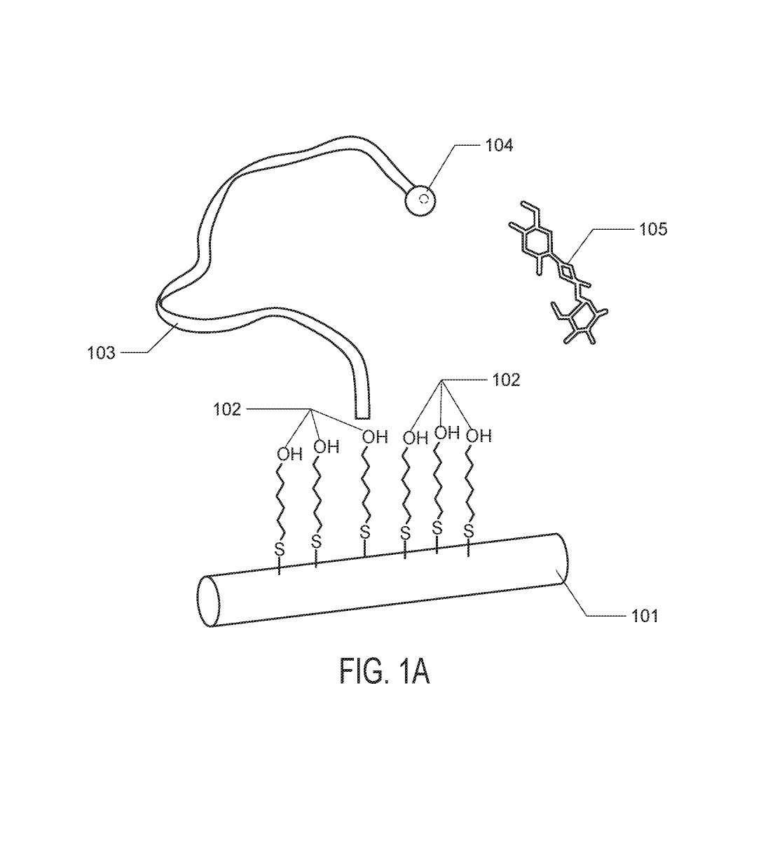

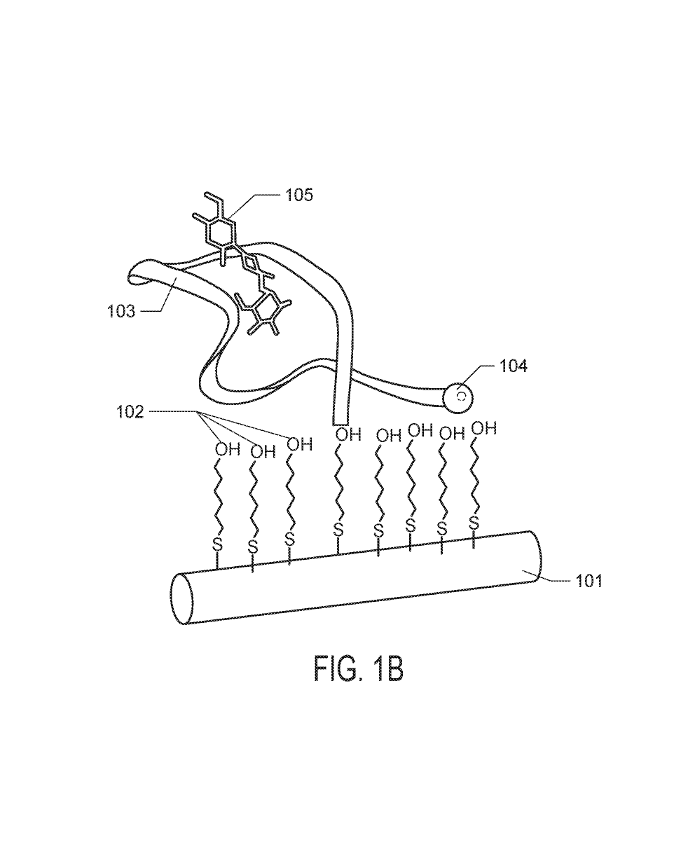

EAB Sensors. EAB sensors are known in the art, for example as described in: U.S. Pat. No. 8,003,374 by Heeger, Fan, and Piaxco; Ferguson et al., “Real-time, aptamer-based tracking of circulating therapeutic agents in living animals,” Sci Transl Med. 2013 Nov. 27; 5(213): 213ra165; and Swensen et al., “Continuous, Real-Time Monitoring of Cocaine in Undiluted Blood Serum via a Microfluidic, Electrochemical Aptamer-Based Sensor,” J Am Chem Soc. 2009 Apr. 1; 131(12): 4262-4266. An exemplary EAB sensor is depicted in FIG. 1A and FIG. 1B. The EAB design comprises various elements, including a working electrode comprising an electrically conducting substrate (101), functionalization moieties that enable functionalization of the substrate (102), a recognition element such as an aptamer (103), and a redox label (104). The recognition element is capable of selectively and reversibly binding a target species (105). As depicted in FIG. 1A, when the target is unbound, the recognition element is free to move and the redox label maintains an average position that is of sufficient distance from the substrate that there is little or no Faradic current or other detectable electronic interaction between the redox label and the substrate. As depicted in FIG. 1B, when the target species is bound to the binding partner, the binding partner assumes a conformation such that the redox label is in proximity to the substrate, causing the flow or Faradic current or other measurable electronic interactions. In a sensor comprising a plurality of recognition elements, the bulk dynamics of target binding and dissociation and the resulting electronic interactions with the substrate create a measurable electronic signal that is proportional to the concentration of the target species in the sample solution.

EAB sensors comprise one or more working electrodes to which recognition elements functionalized with redox labels are bound. The one or more electrodes may comprise various materials and configurations. The electrode may comprise any suitable electrode material for electrochemical sensing, including, for example: gold or any gold-coated metal or material, titanium, tungsten, platinum, carbon, aluminum, copper, palladium, mercury films, silver, oxide-coated metals, semiconductors, graphite, carbon nanotubes, and any other conductive material upon which biomolecules can be conjugated.

The electrode may be configured in any desired shape or size, including discs, strips, paddle-shaped electrodes, rectangular electrodes, electrode arrays, screen-printed electrodes, and other configurations. For in vivo measurements, a thin wire configuration is advantageous, as the low-profile wire may be inserted into cells, veins, arteries, tissue or organs and will not impede blood flow in blood vessels or cause substantial damage in tissues, for example, a wire having a diameter of 1 to 500 μm.

The electrodes of the invention are utilized in sensing systems, which comprise further elements, including counter electrode and/or a reference electrode, a voltage and/or current source, control elements, and readout circuitry, as known in the art. The sensors of the invention can be configured for various electrochemical interrogation techniques, including cyclic voltammetry, differential pulse voltammetry, alternating current voltammetry, square wave voltammetry, potentiometry or amperometry.

The EAB sensor will comprise a plurality of recognition elements. The recognition element comprises a species capable of selectively binding a target molecule, wherein such binding will cause a conformational change in the recognition element or a portion thereof. The recognition element may comprise a nucleic acid (natural or unnatural protein, polysaccharide, non-biological polymer, small molecule, or be of hybrid composition.

In one embodiment, the recognition element is a nucleic acid aptamer. Aptamers are known in the art and may be specific for almost any target, for example being generated by systematic evolution of ligands by exponential enrichment (SELEX) methodologies. DNA aptamers, RNA aptamers, and aptamers comprising non-natural nucleic acids may be used, as well as hybrids of the foregoing. Typical aptamers are about 15-60 bases in length, however, aptamers of any size may be used. Extant aptamers known in the art include those capable of binding target species such as doxorubicin, lysozyme, thrombin, HIV trans-acting responsive element, herein, interferon, vascular endothelial growth factor, prostate specific antigen, dopamine, and cocaine.

The EAB will further comprise an anchoring moiety, which is a chemical species that facilitates attachment of the recognition element to the working electrode. For example, the species comprising the recognition element may be modified at one terminal end with an anchoring moiety. The anchoring moiety may comprise a species which is capable of directly conjugating to the electrode surface, for example by covalent bonding, ionic bonding, adsorption, coordination chemistry or other interaction. Alternatively, the species may be capable of conjugation to a complementary functional group with which the electrode surface has been modified or decorated. Anchoring moieties may comprise elements which form self-assembled monolayers on the electrode surface.

In one embodiment, the anchoring moiety comprises a 3-11 carbon alkyl chain, for example, a six-carbon alkyl chain, the alkyl chain having with a thiol head group, wherein the recognition element is connected at one terminus to the non-thiolated end of the alkyl chain. For example, if the recognition element is an aptamer, the alkyl-thiol chain may be connected at the aptamer 5′ or 3′ terminus or at one or more of the internal bases. Alternatively, the anchoring moiety may comprise a click chemistry group, as known in the art, which is capable of forming bonds with complementary click chemistry groups conjugated to the electrode surface. Alternatively, the anchoring moiety may be an activated silane, as known in the art, which is capable of forming bonds to many oxide surfaces. In another implementation, the anchoring moiety may contain a ligand, which can bind to the surface via coordination bond.

The EAB sensor will further comprise a redox label capable of electron transfer to or from the electrode. With sufficient proximity and accessibility of a redox label to the electrode, an electrical signal, e.g. current, voltage, or other measurable electrical interaction, will occur between the redox label and the electrode. The redox label may be positioned on the recognition element such that binding of the target species to the recognition element causes a measurable change in the electrical signal generated by the redox label. In one embodiment, the redox label is positioned at the terminus of the recognition element, for example as depicted in FIGS. 1A and 1B. In an alternative embodiment, the redox label is present on a separate polynucleotide strand that binds to the aptamer in the absence of target species and that is displaced by binding of the target species to the aptamer, for example as described in Xiao et al., “A Reagentless Signal-On Architecture for Electronic, Aptamer-Based Sensors via Target-Induced Strand Displacement,” J. Am. Chem. Soc., 2005, 127 (51), pp 17990-17991. Redox labels may be configured for turn-off, in which the signal is decreased by the binding of the target species, or turn-on sensing, in which signal is increased by the binding of the target species as known in the art. The placement of such sensing label can be selected using known methods of designing electrochemical sensors.

Exemplary redox labels include methylene blue, ferrocene, viologen, anthraquinone or any other quinones, daunomycin, organo-metallic redox labels, for example porphyrin complexes or crown ether cycles or linear ethers, ruthenium, bis-pyridine, tris-pyridine, bis-imidizole, cytochrome c, plastocyanin, and ethylenetetracetic acid-metal complexes.

EAB sensor fabrication is performed as known in the art. Electrode surfaces may be prepared (e.g. polished, roughened) as known in the art. The electrode surfaces are then functionalized by exposure to a solution comprising the recognition element under conditions which promote the conjugation of the constructs to the electrode. The quantity and density of binding species deposited onto the electrode may be any that is capable of generating measurable sensing and correction signals. For example, densities of between 1×10⁹ to 1×10¹³ molecules/cm² may be used. After the electrode surface has been functionalized with recognition and signaling constructs, additional steps may be performed wash away unbound constructs and/or to passivate exposed electrode surface sites, in order to reduce non-specific interactions with sample constituents.

The scope of the invention includes sensors directed to a single target species, and also includes sensors which are capable of detecting two or more target species. Sensors may be configured with multiple, independently-addressable electrodes to enable multiplexed sensing of two or more target species.

Filtering Encasements to Prevent Fouling. When sensing elements, for example, sensing elements in EAB sensors, are exposed to whole blood or other complex samples, fouling species present in the sample will bind specifically or non-specifically to the sensing element surface. In whole blood, for example, red blood cells, white blood cells, platelets, and other complex macromolecular bodies aggregate and/or coagulate at the sensing element surface. These undesirable interactions cause erroneous signal that increases in a time dependent manner as the degree of non-specific binding increases. This signal drift creates errors when attempting to measure the concentration of the target species in the sample.

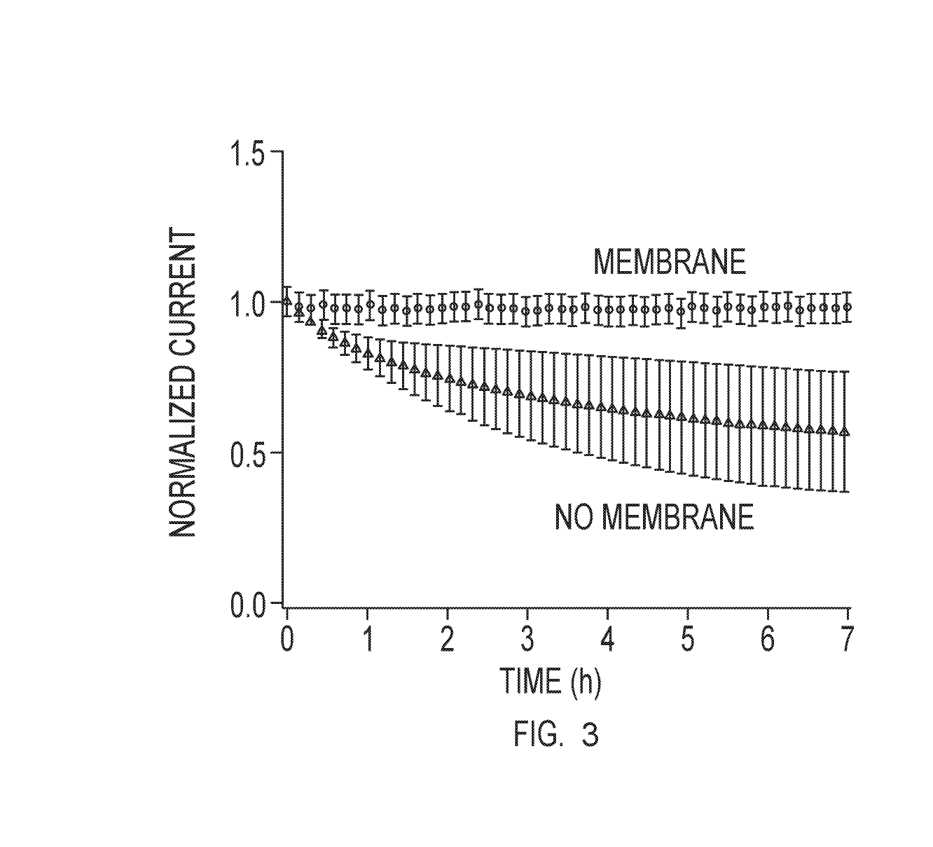

The scope of the invention encompasses the novel use of microporous materials to protect sensing elements from fouling when exposed to whole blood or other complex samples. In the sensors of the invention, the sensing element is encased, surrounded, or otherwise covered by a microporous structure that excludes fouling species. The use of such protective encasements allows, for the first time, real-time, accurate and continuous measurement of diverse target species' concentrations in vivo over extended periods of time. FIG. 3 depicts two EAB kanamycin sensors exposed to whole blood over a period of several hours. One sensor has no protective encasement (labeled “no membrane”) while the other sensor comprises a sensing element covered by a filtering encasement of the invention (labeled “membrane”). As the unprotected EAB sensor becomes increasingly fouled over time, the output signal drifts, decreasing over time. Meanwhile, the output signal of the protected sensor remains stable for hours.

The encasement is sufficiently porous that it allows the liquid comprising the sample, and small target species contained within, to contact the sensor. Simultaneously, the pore size of the encasements is small enough to filter out larger fouling species present in the sample.

Porosity is a measure of the accessible (from the surface of the material) empty or void space within the material, with higher values denoting a greater amount of empty, interconnected space. The porosity of the microporous material may vary, for example being between 10 and 80%. In one embodiment, the microporous material has a porosity of 25 to 35%. Sufficient porosity is required for the free exchange of fluids between the bulk sample and the layer of fluid that is in contact with the sensing element. This ensures that the fluid in contact with the sensing element is not isolated from the bulk fluid being sampled, such that real-time changes in the concentration of target species in the bulk sample are detectable. In general, higher porosity values are desirable in order to facilitate real-time exchange of sample fluid between the sensor surface and the bulk sample, avoiding a localized depletion zone around the sensing element, which will lead to erroneous measurements and decreased time resolution. However, excessive porosity will weaken the structural integrity of the material and porosity will need to be balanced against the durability requirements of the sensing assembly.

“Pore size,” as used herein will refer to the size exclusion limit of the encasement, i.e. the maximum size of species that can pass through the encasement material in measurable quantities. In some embodiments, the material comprising the encasement will have defined holes or pores. In other embodiments, the material lacks defined pores, but is discontinuous, for example in the case of spongy or fibrous materials. The pore size of the encasement will be selected based on the nature of fouling species present in the sample and the size of the target species. :For most biological and environmental applications, pore sizes between 50 nm and 4 μm may be used. In one embodiment, the pore size of the encasement material is between 100 nm and 1 μm. When used in human blood, for example, a pore size of greater than 50 nm and less than 2 μm in diameter is appropriate, for example a pore size of 200 nm.

The filtering encasements of the invention may comprise various materials. In one embodiment, the encasement comprises polysulfone (polyethersulfone). The pore size and pore density of polysulfone membranes may be tuned, as known in the art, for example as described in Ficai et al., 2010, Polysulfone based Membranes with Desired Pores Characteristics, Material Plastice 47: 24-27 and Ulbricht, 2006, Advanced Functional Polymer Membranes, Single Chain Polymers 47: 2217-2262.

Additional exemplary encasement materials include microporous, poly-tetrafluoroethylene (i.e., GORE-TEX™), polyether-urethaneurea (Vectra™) and polyethylene terephthalate (Dacron™).

In biological applications it is generally desirable that the material comprising the encasement is biocompatible and/or biologically inert. In some applications it is desirable that the encasement comprise a flexible material in biological applications, especially for the deployment of sensing elements in whole blood, the encasement material may be modified to increase its resistance to coagulation, for example by functionalization with PEG, heparin, or citrate molecules at sufficient density to inhibit coagulation.

The encasements of the invention may be of any size and shape and is generally matched to the size and shape of the sensing element. The encasement may be configured as a hollow body having an interior portion and exterior portion, wherein a sensing element is placed within the interior portion of the encasement and the interior portion is sealed off from the surrounding environment. For example, if the functionalized electrode comprises a wire, the encasement may comprise a tubular body, into which the wire is inserted, having an internal lumen that is the same diameter of the wire or slightly larger. In other embodiments, the encasement may comprise a patch which covers the sensing element. A small headspace may be present between the interior surface of the encasement and the sensing element, or the encasement may be flush against the sensing element.

The edges or openings of encasement are sealed around the sensing element to prevent leakage of fouling species into the sensing area. The encasement may be held in place around the sensing element by any means, including by use of fasteners, adhesives, tension forces or other mechanical structures/forces.

The combination of the sensing element and microporous structure surrounding it will be referred to herein as a “sensor assembly.” The scope of the invention encompasses sensing assemblies capable of operation in complex samples such as blood. The scope of the invention further encompasses methods of using porous filters to protect sensing elements from fouling species. In one embodiment, the sensor is an EAB sensor. In one embodiment the sample is blood, for example blood within a living organism. In another embodiment, the fouling species is one or more of red blood cells, white blood cells, platelets and other macromolecular species present in blood that can cause coagulation at and/or fouling of an electrochemical surface.

Sensor Configuration. The sensor assembly may be configured in any desired shape or size. In some embodiments, the sensor may comprise an in vivo probe or implant, as described below. In some embodiments, the sensor comprises a tabletop lab apparatus. In other embodiments, the sensor comprises a hand-held device. In other embodiments, the sensor comprises a microfluidic biochip.

In one embodiment, the sensor of the invention is configured as an in vivo sensor. An “in vivo” sensor means a sensor configured to sample fluids within the body of a living organism. When an in vivo sensor is in use, the sensing assembly is inserted, implanted, or otherwise placed within the body of a living organism such that the sensing element is exposed to in-vivo fluids, e.g. blood. In one embodiment, only the sensing assembly or a portion thereof is located within the body of the living organism and is in connection (e.g. by wires) with other sensor elements which are located outside of the body of the living organism. In one embodiment, the sensor is a wearable sensor comprising external components strapped, adhered, or otherwise held in place outside the body and further comprising a sensing assembly placed in vivo. Alternatively, some all of the ancillary sensor components may be placed within the body, for example in the case of highly miniaturized, implanted devices.

For in vivo measurements, a sensing assembly comprising a thin wire configuration is advantageous, as the low-profile wire may be inserted into veins, arteries, tissue or organs and will minimally impede blood flow in blood vessels or will cause minimal damage in the sampled area. For example, a wire having a diameter of 1-500 μm, for example, 100 μm, may be used.

In one embodiment, the sensing assemblies are housed in a needle, catheter, or cannula which may be inserted into a vein, blood vessel, organ, tissue, or interstitial space in order to place the sensor in the target environment. The needle, catheter, or cannula may be porous, comprising a plurality of holes or channels distal to the tip in order to allow the flow of blood over the sensor assembly. Alternatively, the sensing element may be placed on a supporting body that can be extended from and retracted into the needle, catheter, or cannula to protect it during insertion and then deploy it into the bloodstream or other inter compartment of the animal, placing it in contact with the sample fluid.

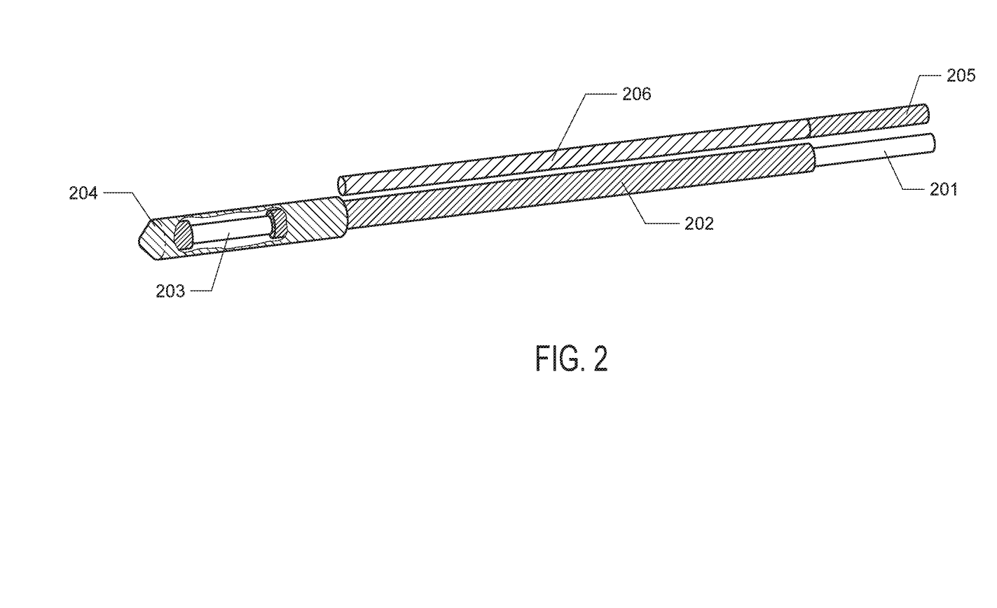

An exemplary wire sensor configuration is depicted in FIG. 2. FIG. 2 depicts an EAB sensor comprising an elongated wire working electrode (201). The non-sensing portion of the wire is coated with an insulating material (202). The sensing portion of the wire (203) is housed beneath a filtering encasement (204-cut away to show 203 underneath). This working electrode is paired with a reference electrode comprising a wire (205), the reference electrode wire optionally being coated with an oxide layer or other material (206).

Applications of the Sensors of the Invention. The novel sensors of the invention may be utilized in many contexts. In a first aspect, the scope of the invention encompasses any utilization of the sensors of the invention to measure the concentration of a target species in a sample. The sample may comprise blood, serum, interstitial fluid, spinal fluid, cerebral fluid, tissue exudates, macerated tissue samples, cell solutions, intracellular compartments, groundwater, or other biological and environmental samples. Samples may be unaltered or may be pretreated prior to analysis, for example being filtered, diluted, concentrated, buffered, or otherwise treated.

Measurement of the target species may be accomplished by any means amenable to the selected sensing element. For example, if the sensor is an EAB sensor, the target species may be assayed by methodologies such as cyclic voltammetry, differential pulse voltammetry, alternating current voltammetry, square wave voltammetry, potentiometry or amperometry. In one embodiment, the use of kinetic differential measurement techniques, as known in the art can be employed to improve signal to noise ratio.

The sensors of the invention may be used in in-vivo applications. In one embodiment, the method of the invention comprises the steps of inserting a sensing assembly of the invention into a selected area of a living organism and measuring target species concentration at the target site over time. In one embodiment, the selected area of the body is in the circulatory system, e.g. in a vein or blood vessel, wherein the sensor is exposed to a continuous flow of whole blood. In alternative embodiments, the sensor may be placed subcutaneously, intramuscularly, or within a target organ. In one embodiment, the in vivo sensor comprises a wire electrode configuration.

The sensors of the invention may also be used in ex-vivo applications. In one embodiment, the method of the invention comprises the steps of withdrawing a sample from a living organism, exposing a sensor of the invention which is directed to detection of a target species to the sample, and measuring the concentration of the target species in the sample. In one embodiment, the sample fluid is withdrawn continuously from the living organism and target species concentration is measured on a prolonged basis. In one embodiment, a single sample is analyzed. In one embodiment, the sample is blood. In one embodiment, the sensor is housed in a wearable or otherwise portable device.

In one embodiment, the sensors of the invention are employed in point of care testing methods. In such an application, a sample is withdrawn from the patient and the concentration of a target species is measured using a sensor of the invention. For example, in one embodiment, the sample is a blood sample, for example, a pin-prick or finger-prick blood sample, for example, a self-withdrawn pin-prick or finger-prick blood sample. The sensors of the invention advantageously enable the immediate testing of small blood samples, obviating the need for processing the blood sample prior to analysis.

In one embodiment, the sensors of the invention are used to monitor the concentration of a target species in a living organism over time, for example, for periods of minutes, to hours, to several days. In one embodiment, the living organism is a patient and the target species is a drug.

Pharmacokinetic Measurements. Generally, it is desirable to maintain the concentration of a drug within a patient within an optimal range. Under-dosing will result in ineffective treatment. Excessive dosages may result in harmful or undesirable side effects, as well as significant costs in the case of expensive agents. Accordingly, there is a need in the medical arts to create efficient dosing regimes for patients that maintain the concentration of the drug within the optimal range. The sensors and associated methods of the invention provide the art with tools for determining optimal dosage regimes, at both the individual and population level.

In one embodiment, the sensors of the invention enable personalized pharmacokinetic parameters to be established in an individual patient. The pharmacokinetics of a drug are known to vary widely among patients, due to personal differences in metabolism, enzymatic activity, etc. Accordingly, a dosage regime which maintains a drug's optimal concentration in one patient will likely be non-optimal in some other patients. As described in the Example section below, the sensors of the invention are sensitive enough to detect significant variability in drug metabolism between individual animals administered identical dosages of a drug.

Accordingly, the sensors of the invention allow for the determination of pharmacokinetic parameters in an individual with respect to a specific drug. In such a method, a sensor of the invention capable of measuring the concentration of the selected drug is deployed within a subject animal, e.g. a patient. For example, the sensor may be deployed to the circulatory system to monitor blood levels of the drug on a continuous basis. Next, one or more doses of the drug, is administered. Subsequently, the concentration of the drug in the subject is monitored over time (e.g. minutes, hours, days). The concentration vs. time data generated thereby may then be subsequently analyzed, using tools known in the art, to calculate distribution and elimination profiles for the subject, or other pharmacokinetic parameters. These observations can be used to construct a personalized dosage regime for the individual that maintains the drug's concentration within the optimal range.

Likewise, the afore-described pharmacokinetic analyses can be performed in a plurality of subjects within a population. Data generated therefrom may be used to construct a generalized dosing regime for members of the population.

Drug Delivery. In another embodiment, the sensors of the invention enable feedback controlled dosing systems. The concept of feedback controlled dosing is known in the art, for example as reviewed by LeVan et al., “Small-scale systems for in vivo drug delivery.” Nature Biotechnology 21, 1184-1191 (2003), with various exemplary implementations described in U.S. Pat. No. 5,697,899, entitled “Feedback controlled drug delivery system,” to Hillman, and U.S. Pat. No. 7,108,680, entitled “Closed loop drug delivery system” to Rhor.

The basic concept of feedback controlled drug delivery is the automated administration of a drug to the user based on real-time measurement of the drug's concentration in the body, wherein an aliquot of drug is administered when it is detected that the blood level of the drug has dropped below a selected threshold. Alternatively, feedback controlled dosing can be based upon the concentration of a drug-associated species in the patient. A drug-associated species is a chemical marker or biomarker that is indicative of the concentration of the drug in the patient or which is indicative of the need for administration of the drug to the patient. An existing example of feedback controlled drug delivery based on a drug-associated species is the implantable insulin pump, wherein insulin (the drug) is administered in response to real-time measurements of blood glucose (the drug-associated species).

Feedback controlled dosing would provide the medical arts with a superior means of treating patients, allowing a drug's concentration in the body to be perfectly maintained within the optimal therapeutic range. Despite the potential benefits that feedback controlled drug delivery systems could provide, actual adoption of the concept has been limited, because of the lack of reliable in-vivo sensors that can operate in whole blood. Accordingly, the sensors and methods of the invention provide a novel and versatile platform technology that enables widespread implementation of feedback controlled drug delivery for a wide array of therapeutics and conditions.

In application, a patient in need of treatment is administered a selected drug. The timing of drug delivery will be based on the measured concentration of the drug in the body of the patient, or on the concentration of a drug-associated species. Thresholds concentrations are selected that trigger drug delivery, for example, “deliver more drug if the concentration of the drug drops below concentration X” or “administer more drug if the concentration of biomarker X exceeds concentration Y.” Next, an implanted sensor of the invention is utilized to continuously measure the concentration of the drug or selected drug-associated species within the patient. When the concentration of the target species meets the selected threshold, drug delivery is triggered. A device coupled with or in communication with the sensor, for example comprising an implanted pump or other drug delivery means, is engaged to administer an aliquot of the drug sufficient to maintain the concentration of the drug within the optimal range or to otherwise treat the patient's condition. Alternatively, when the concentration of the target species meets the selected threshold, a device coupled with or in communication with the sensor can be engaged to alert medical personnel or the patient, who can subsequently administer, or self-administer, an aliquot of the drug (e.g. orally) to restore or maintain the concentration within the optimal range.

The scope of the invention encompasses methods of feedback controlled dosing utilizing sensors of the invention. The scope of the invention further encompasses devices for the implementation of feedback controlled dosing, comprising sensors of the invention coupled with or in communication with drug delivery devices such as implantable pumps or other drug delivery devices known in the art. In another embodiment, the invention comprises a sensor of the invention coupled with or in communication with a device that can alert the user or medical personnel when the concentration of a the target species meets the selected threshold, for example, a device which displays a concentration value or an alert message or a device which plays an audible tone.

### EXAMPLES

Materials and Methods. Sensors were constructed as previously described, for example, as in White, R. J. and Plaxco, K. W. (2010) “Exploiting binding-induced changes in probe flexibility for the optimization of electrochemical biosensors.” Anal. Chem., 82, 73-76. EAB sensors were then fitted with the novel filtering encasements of the invention.

Methylene-blue-and-thiol-modified aptamers directed to tobramycin, doxorubicin, and aminoglycoside were used in various experiments. The 5′ end of each was modified with a thiol on a 6-carbon linker and the 3′ end was modified with carboxy-modified methylene blue attached to the DNA via the formation of an amide bond to a primary amine on a 7-carbon linker. The length of the surface tethering carbon linker represents a compromise between the two main criteria for electrochemical biosensor applications: stability and electron-transfer efficiency. A 6-carbon linker was selected because it exhibits good stability and improved signaling relative to that seen, for example, when using 11-carbon linkers. The modified DNAs were purified through dual HPLC by the supplier and used as received. Upon receipt each construct was dissolved to 200 μM in 1× Tris-EDTA buffer and frozen at −20° C. in individual aliquots until use.

Silver wire (200 μm diameter) was used to construct the reference electrode for each sensor. It was immersed in bleach overnight to form a silver chloride film. Gold-plated tungsten wire (100 μm diameter) was used as the working electrode. Polyethersulfone membranes (P/N: C02-E20U-05-N) were purchased as MicroKros™ Filter Modules from Spectrum Laboratories (Rancho Dominguez, Calif.). The filter modules were cut open and the hollow membranes were extracted from them. Heat-shrink polytetrafluoroethylene insulation (PTFE, HS Sub-Lite-Wall, 0.02, 0.005, 0.003±0.001 in, black-opaque, Lot #17747112-3) was used on gold-plated tungsten.

Segments of either gold-plated tungsten wire (anesthetized animals) or more malleable pure gold wire (awake animals) 7 cm in length were cut to make sensors. These wires were then insulated by applying heat to shrinkable tubing around the body of the wires, as depicted in FIG. 2. The sensor window (i.e., the region without insulation) was approximately 5-8 mm in length. To increase surface area of these working electrodes (to obtain larger peak currents) the sensor surface was roughened electrochemically via immersion in 0.5 M sulfuric acid by jumping between Eᵢₙᵢₜᵢₐₗ=0.0 V to E_{high}=2.0 V vs Ag/AgCl, back and forth, for 100,000 pulses. Each pulse was of 2 ms duration with no “quiet time.”

To fabricate sensors an aliquot of the appropriate DNA construct was thawed and then reduced for 1 h at room temperature with a 1000-fold molar excess of tris(2-carboxyethyl)phosphine. A freshly roughened gold electrode was then rinsed in di-ionized water before being immersed in a solution of the appropriate reduced DNA construct at 200-500 nM in PBS for 1 h at room temperature. Following this the sensor was inserted into hollow polysulfone fibers 1.5 cm in length and 200 μm in diameter. The membranes were mechanically attached to the sensors by wrapping the edges with Parafilm™. After attaching the membranes, the sensors were immersed overnight at 4° C. for 12 h in 20 mM 6-mercapto-1-hexanol in PBS to coat the remaining gold surface and remove nonspecifically adsorbed DNA. After this the sensors were rinsed with di-ionized water and stored in PBS.

Electrochemical Methods and Data Processing. For all sensing experiments, the sensors were interrogated using square wave voltammetty from 0.0 V to −0.5 V vs, AglAgCl, using an amplitude of 50 mV, potential step sizes of 1-5 mV, and varying frequencies from 10 Hz to 500 Hz. The files corresponding to each voltammogram were recorded in serial order using macros in CH Instruments software.

All in vitro measurements were performed using a three-electrode setup and with a CH Instruments electrochemical workstation (Austin, Tex., Model 660D) using commercial Ag/AgCl reference electrodes filled with saturated KCl solution and platinum counter electrodes.

All in vivo measurements were performed using a two-electrode setup in which the reference and counter electrodes were a silver wire coated with a silver chloride film as described above. The measurements carried out in vivo were recorded using a handheld potentiostat.

In vitro Experiments. To measure aptamer affinity and correlate signal gain to target concentration, sensors were interrogated by square-wave voltammetry first in flowing PBS and next in flowing heparinized human or bovine blood with increasing concentrations of the corresponding target. These experiments were carried out in a closed flow system intended to mimic the type of blood transport found in veins. Blood flow was achieved using a magnetic gear pump (0.261 mL/rev), setting flow rates to 1-4 mL, min⁻¹ as measured by a flow meter. To construct the binding curves (titrations of aptamer with target), stock solutions of each target molecule were prepared fresh prior to measurements in PBS buffer or blood, respectively.

In vivo Experiments Animals. Adult male Sprague-Dawley rats (300-500 g) were pair housed in a temperature and humidity controlled vivarium on a 12-h light-dark cycle and provided ad libitum access to food and water. All animal procedures were consistent with the guidelines of the NIH Guide for Care and Use of Laboratory Animals.

Surgery. For the anesthetized preparation, rats were anesthetized using isoflurane gas inhalation (2.5%) and monitored throughout the experiment using a pulse oximeter to measure heart rate and % SpO2 to insure depth of anesthesia. After exposing both ventral jugular veins, a simple catheter made from a SILASTIC tube (Dow Corning, Midland, Mich., USA) fitted with a steel cannula was implanted into the left jugular vein. 0.1-0.3 mL of heparin (1000 U/mL) were immediately infused through the catheter to prevent blood clotting. The sensor was inserted into the right jugular vein and secured in place with surgical suture.

For the awake preparation, rats were anesthetized (as above) and then mounted on a stereotaxic apparatus with a gas anesthesia head holder to maintain anesthesia. After a subcutaneous injection of an analgesic (1 mg/kg), a midline incision was made along the dorsal surface of the scalp and a second incision was made on the ventral portion of the neck above the jugular vein. Using a similar catheter construction described above, a catheter tube was implanted into the right jugular vein and sutured it in place before sealing the wound with skin glue. The surface of the skull was then exposed and 4 screws were drilled into the bone to provide a platform for the cannula to be cemented to the head. Dental cement was applied to the skull surface while the cannula was held in place using the stereotaxic arm. After the cement had set, the catheter was flushed with antibiotics (1 mg/kg gentamicin and 1 mg/kg cefazolin) and the animal was monitored for postoperative recovery before being returned to the vivarium colony. Daily monitoring of weight and condition of recovery followed for 4 days in which the animal was treated with analgesic (as above) and observed for signs of distress/wound inflammation. No further procedures were carried out on these animals for a minimum of one week.

Measurements. A 30-minute sensor baseline was established before the first drug infusion. For anesthetized animals, a 3 mL syringe filled with the target drug was connected to the sensor-free catheter (placed in the jugular opposite that in which the sensor is emplaced) and placed in a motorized syringe pump. After establishing a stable baseline, the target drug was infused through this catheter at a rate of 0.2 mL/min. Target drugs included kanamycin (0.1 M solution), gentamicin (10 mg/mL), tobramycin (0.1 M solution), and doxorubicin (1.0 mM). After drug infusion, recordings were taken for up to 2 hours before the next infusion.

For the awake preparation, a pre-catheterized animal was first briefly anesthetized (as above). The sensor was threaded down the catheter and tightly attached to it via a homemade plastic joint. The joint protected the sensor from being accidentally pulled out by the animal while exploring surroundings. Once implanted, the EAB sensor was affixed to a leash in an operant chamber. The animal was then allowed to recover from anesthesia and explore the chamber while recordings proceeded as described above. Following the baseline recording, the target drug was introduced via either an intramuscular injection (thigh) or via an intravenous injection given through the same catheter used to emplace the sensor.

### Results

To reduce fouling, EAB sensors were encased in biocompatible polysulfone membranes, the 0.2-μm pores of which prevent blood cells from approaching the sensor surface while simultaneously allowing for the rapid transport of target molecules.

Using these membranes, stable EAB baselines were achieved in flowing, undiluted whole blood in vitro over many hours. For example, the plot of FIG. 3 shows a comparison of baseline drift between the membrane-modified platform (no current change in 6 hours) and conventional aminoglycoside EAB sensors (40% current loss in 6 hours). Normalized currents correspond to peak currents from square-wave voltammograms divided by the peak current of the first voltammogram.

Even membrane-protected EAB sensors, however, may exhibit some baseline drift when emplaced in the veins of live animals. To circumvent this drift, the Kinetic Differential Measurement correction scheme was applied. Drift correction methods have historically used a physically separate reference that, although unresponsive to the targeted input, nevertheless yields an identical response to background that can be subtracted from the sensor outputs instead employs a single aptamer in both roles, thus obviating the need to fabricate a matched sensor-reference pair. To achieve this stand-alone performance, Kinetic differential measurement exploits the square wave frequency dependence of EAB signaling. Specifically, electron transfer is more rapid from the folded, target-bound aptamer than it is from the unfolded, target-free aptamer. This kinetic difference results in a binding-induced increase in current when square-wave voltammetry is performed at high frequencies and a binding-induced decrease in signal at low frequencies. Conveniently, these two outputs drift in concert, and thus taking their difference effectively corrects baseline drift.

Drift-corrected, membrane-protected. EAB sensors readily support the continuous, seconds-resolved real-time measurement of specific molecules in the blood of living animals. To demonstrate this ability, EAB sensors for the detection of the cancer chemotherapeutic doxorubicin (DOX) were emplaced in the external jugular vein of anesthetized Sprague-Dawley rats. Using this approach, nanomolar precision was achieved in the measurement of clinically relevant plasma drug levels following five sequential injections over 5 hours of continuous monitoring. The resulting plot of concentration versus time presented consecutive spikes corresponding to each of the injections performed, with maximum DOX concentrations (Cₘₐₓ) of ˜600 nM and the effective clearance of 90% of the drug from the circulatory system within 50 min, values in close accord with prior reports.

Sensors were fabricated using an aptamer recognizing the aminoglycoside antibiotics. Using these sensors, monotonically increasing intravenous doses of kanamycin were administered spanning the therapeutic ranges used in humans (10-30 mg/kg) and animals (25-30 mg/kg). The sensor responded rapidly to each injection, measuring maximum concentrations between 34 and 400 μM depending on the delivered dose. The 200 μM maximum concentration observed after a 10 mg/kg dose was in agreement with peak plasma concentrations determined previously (using cumbersome, poorly time-resolved ex vivo radioimmunoassays) after similar doses were injected into multiple animal species. The sensor can likewise monitor in real time the in vivo concentrations of the aminoglycosides gentamycin and tobramycin following either intramuscular or intravenous injections, applications in which it once again achieves excellent precision and time resolution.

The pharmacokinetics of tobramycin were monitored following sequential 20 mg/kg intravenous injections conducted 2 hours apart in each of three rats. FIG. 4 depicts the continuous measurement of the antibiotic tobramycin by a sensor of the invention in the bloodstream of an anesthetized rat. Shown are data collected on a living rat given two sequential 20 mg/kg intravenous injections of the drug (at times denoted by vertical dotted lines), demonstrating the sensor's ability to accurately track target species concentration at short time scales.

Fitting the resultant data to a two-compartment model, significant inter- and even intra-animal variability was observed. The distribution phase (α phase) of this drug, for example, is defined largely by blood and body volume and thus, although the distribution differs between animals, it differs much less as a function of time within individual animals. The elimination kinetics of tobramycin (β phase), in contrast, not only vary significantly between animals but also exhibit variations within a single individual over the course of a few hours that are easily measurable using the approach of the invention. For example, although the kinetics of the α phase remain relatively constant for a given animal, the β phase invariably slows with time. This change presumably occurs because, whereas drug absorption (captured by the α phase) is defined by body volume, which remains fixed, the elimination of tobramycin (captured in the β phase) is predominantly via excretion from the kidneys, the function endogenous metabolites and hormones in rat blood activates the sensor, as evidenced by their performance in vivo.

In addition to studies, as those above, performed on anesthetized animals, the simplicity, physical robustness, and small size of EAB sensors also rendered it possible to perform measurements on awake, ambulatory animals. To illustrate this ability, permanent catheters were surgically implanted in the jugular veins of rats and the animals were allowed to recover from this surgery for 2 weeks before using the catheter to insert a flexible EAB sensor under light anesthesia. The sensor connects to its supporting electronics via flexible wire leads that allow the awake animals to move largely unimpeded. Aminoglycoside sensors used under these conditions support run times of up to half a day as the animal feeds, drinks, and explores its environment producing pharmacokinetic data that avoid potentially confounding factors associated with measurements based on (repeated) blood draws, which require anesthetized or otherwise immobilized (and thus stressed) animals.

In conclusion, the examples presented herein demonstrate the ability of novel sensors of the invention to track specific small molecules continuously in real-time in awake, ambulatory animals.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

What is claimed is:

1-33. (canceled)

34. A sensor for measuring the concentration of a target species in a fluid sample, comprising
a sensing element which generates a signal in response to binding of the target species, wherein the sensing element comprises a sensing element selected from the group consisting of the following: electrode functionalized with aptamers, a surface plasmon resonance sensor, a quartz crystal micro-balance sensor, a field-effect transistor, and a microcantilever-based sensor; and
a microporous encasement;
wherein the sensing element is encased within the microporous encasement, which allows the fluid sample to contact the sensing element while preventing contact between the sensing element and fouling species present in the sample.

35. The sensor of claim 34, wherein
the sensing element comprises an electrode functionalized with aptamers.

36. The sensor of claim 34, wherein
the encasement comprises a material having a porosity of 10-80% and a pore size of between 50 nm and 4 μm.

37. The sensor of claim 38, wherein
the pore size is 100-200 nm

38. The sensor of claim 34, wherein
the encasement comprises polysulfone.

39. The sensor of claim 34, wherein
the encasement comprises a material selected from the group consisting of poly-tetrafluoroethylene, polyether-urethane and polyethylene terephthalate.

40. The sensor of claim 34, wherein
the encasement is functionalized with species that prevent the coagulation of blood.

41. The sensor of claim 34, wherein
the sensing element and encasement comprise an elongated wire configuration with a diameter between 1 to 500 μm.

42. The sensor of claim 34, wherein
the sensing element and encasement are housed in a needle, catheter, or cannula.

43. A drug monitoring system, comprising
a sensor for measuring the concentration of a target species in a fluid sample, comprising a sensing element which generates a signal in response to binding of the target species, wherein the sensing element comprises a sensing element selected from the group consisting of the following: electrode functionalized with aptamers, a surface plasmon resonance sensor, a quartz crystal micro-balance sensor, a field-effect transistor, and a microcantilever-based sensor; and a microporous encasement;
wherein the sensing element is encased within the microporous encasement, which allows the fluid sample to contact the sensing element while preventing contact between the sensing element and fouling species present in the sample; and
a secondary device coupled with or in communication with the sensor, wherein the secondary device is activated when the concentration of the target species detected by the sensor meets or passes a selected threshold value.

44. The drug monitoring system of claim 43, wherein
the encasement comprises polysulfone, poly-tetrafluoroethylene, polyether-urethane or polyethylene terephthalate.

45. The drug monitoring system of claim 43, wherein
the secondary device comprises a device which issues an alert when activated.

46. The drug monitoring system of claim 45, wherein
the sensor is implanted in a patient and the alert is received by the patient.

47. The drug monitoring system of claim 43, wherein
the secondary device comprises a device which administers a selected aliquot of an agent to an animal when it is detected that the concentration of the target species rises above or falls below a selected threshold.

48. A method of measuring the concentration of a target species in a sample, comprising
utilizing a sensor to measure the concentration of the target species in the sample, wherein the sensor comprises
a sensing element which generates a signal in response to binding of the target species, wherein the sensing element comprises a sensing element selected from the group consisting of the following: electrode functionalized with aptamers, a surface plasmon resonance sensor, a quartz crystal micro-balance sensor, a field-effect transistor, and a microcantilever-based sensor; and
a microporous encasement; and
wherein the sensing element is encased within the microporous encasement, which allows the fluid sample to contact the sensing element while preventing contact between the sensing element and fouling species present in the sample.

49. The method of claim 48, wherein
the sensing element and filtering encasement of the sensor is inserted, implanted, or otherwise present in the body of a living organism.

50. The method of claim 49, wherein
the sample is whole blood;
the living organism is an animal; and
the sensing element is inserted, implanted, or otherwise present in the circulatory system of the animal.

51. The method of claim 48, wherein
the encasement comprises polysulfone, poly-tetrafluoroethylene, polyether-urethane or polyethylene terephthalate.

52. The method of claim 48, comprising the additional step of
administering an aliquot of a selected agent to the animal when the concentration of the target species falls below or rises above a selected threshold.

53. The method of claim 48, comprising the additional step of
issuing an alert when the concentration of the target species falls below or rises above a selected threshold.
